(19) United States
(12) Patent Application Publication
(10) Pub. No.: US 2024/0307577 A1
(43) Pub. Date: Sep. 19, 2024

(54) CHLORINATED 4'-ALKYLUMBELLIFERYL ALPHA-D-GLUCOPYRANOSIDE AND METHOD OF ASSESSING EFFICACY OF A STERILIZATION PROCESS INCLUDING THE SAME

(71) Applicant: 3M INNOVATIVE PROPERTIES COMPANY, St. Paul, MN (US)

(72) Inventors: Stephen B. Roscoe, Woodbury, MN (US); George W. Griesgraber, Eagan, MN (US); Tonya D. Bonilla, Woodbury, MN (US); Giuseppe M. Bommarito, Stillwater, MN (US); Wensheng Xia, Woodbury, MN (US); Jeffrey M. Flynn, Osceola, WI (US)

(21) Appl. No.: 18/576,785

(22) PCT Filed: Jul. 18, 2022
(86) PCT No.: PCT/IB2022/056605

§ 371 Date: Jan. 5, 2024

Related U.S. Application Data
(60) Provisional application No. 63/224,625, filed on Jul. 22, 2021.

(51) Int. Cl. A61L 2/28; A61L 2/16; C07H 17/075; C09K 11/06; C12Q 1/22

(52) U.S. Cl. CPC A61L 2/28; A61L 2/16; C07H 17/075; C09K 11/06; C12Q 1/22

(57) ABSTRACT

A chlorinated 4′-alkylumbelliferyl α-D-glucopy-ranoside represented by the structural formula (I) One of R¹ and R² is Cl and the other is H. R³ represents an alkyl group having 1 to 12 carbon atoms. A method of assessing efficacy of a sterilization process including the same is also disclosed.

### TECHNICAL FIELD

The present disclosure broadly relates to chlorinated 4′-alkylumbelliferyl α-D-glucopyranosides, biological sterilization indicators including the same and methods of using the same.

### BACKGROUND

Biological sterilization indicators (biological indicators) provide a means for determining whether a sterilizing machine, such as those used to sterilize surgical instruments in hospitals, is functioning properly and killing microorganisms that are present in the sterilization chamber during a sterilization procedure.

Biological indicators are recognized in the art as providing an accurate and precise means for testing the effectiveness of a sterilization procedure. In contrast to sterilization indicators that measure spore outgrowth alone, biological indicators that measure the activity of an enzyme whose activity is correlated with the destruction of contaminating microorganisms during a sterilization procedure provide a faster result. For example, Geobacilllus stearothermophilus α-glucosidase activity correlates with the loss or maintenance of spore viability post-sterilization, which provides test results within minutes to hours instead of days (growth-based detection readings).

In one method, the substrate 4′-methylumbelliferyl-α-D-glucopyranoside (MUG, shown below):

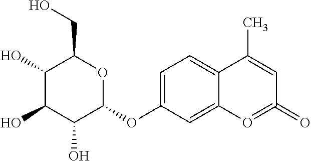

is incorporated into a detection medium. Once in contact with the medium, a spore-derived enzyme rapidly hydrolyzes the substrate to release the intensely fluorescent molecule 4-methylumbelliferone, shown below.

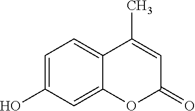

The deprotonated form of 4-methylumbelliferone (pkₐ=7.8), shown below:

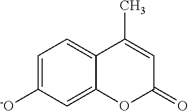

Highest fluorescence is observed in its deprotonated anionic form, typically having a greater fluorescence intensity at pH=10 than at a pH of 6-8, when excited at 360 nanometers (nm), with an emission maximum of 455 nm. However, a pH of 6-8 is typically desired for the detection of α-glucosidase activity from Geobacillus stearothermophilus endospores.

Detection of α-glucosidase activity is often preferred for detection of G. stearothermophilus in biological indicators used to monitor sterilization protocols; for example, as described in U.S. Pat. No. 5,073,488 (Matner et al.).

Using MUG substrate, the enzyme can be detected within short time-frames from fractional kill sterilization cycles. Another significant advantage of using this system for biological sterilization indicators, in particular self-contained steam sterilization biological sterilization indicators, is that MUG substrate can tolerate high heat exposures.

PCT Publications WO 2020/115661 A1 (Bonilla et al.) and WO 2021/059058 A1 (Roscoe et al.) disclose certain fluorinated 4′-alkylumbelliferyl α-D-glucopyranosides that can be used in methods of assessing efficacy of a sterilization process.

### SUMMARY

Advantageously, the chlorinated 4′-alkylumbelliferyl α-D-glucopyranosides disclosed herein are easily prepared and exhibit a higher fluorescent signal, at high and low spore levels, in the presence of Geobacillus stearothermophilus spores than the traditional enzyme substrate 4′-methylumbelliferyl α-D-glucopyranoside and 8′-fluoro-4′-methylumbelliferyl α-D-glucopyranoside.

Accordingly, in one aspect, the present disclosure provides a chlorinated 4′-alkylumbelliferyl α-D-glucopyranoside represented by the structural formula

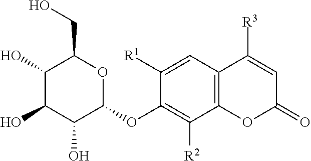

wherein:

one of R¹ and R² is Cl and the other is H; and

R³ represents an alkyl group having 1 to 12 carbon atoms.

In another aspect, the present disclosure provides a method of assessing efficacy of a sterilization process, the method comprising sequentially:

a) providing a biological sterilization indicator comprising:
i) bacterial spores comprising, and/or capable of producing, an enzyme capable of catalyzing cleavage of an enzyme substrate represented by the structural formula

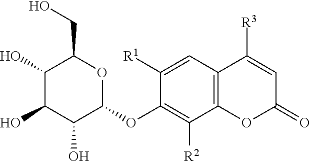

wherein:
   one of R¹ and R² is Cl and the other is H, and
   R³ represents an alkyl group having 1 to 12 carbon atoms; and
ii) a composition, wherein the composition comprises the enzyme substrate, wherein if the composition is brought into contact with the bacterial spores to form a mixture, the mixture will have an initial pH in the range from 5.5 to 9.0;

b) subjecting at least the bacterial spores to the sterilization process;

c) contacting the composition with the bacterial spores; and

d) evaluating efficacy of the sterilization process.

In some preferred embodiments, step d) comprises fluorescence spectroscopy.

Features and advantages of the present disclosure will be further understood upon consideration of the detailed description as well as the appended claims.

### DETAILED DESCRIPTION

The present disclosure provides monochlorinated 4′-alkylumbelliferyl α-D-glucopyranosides, which can be used as enzyme substrates, that are stable under typical thermal conditions used in sterilization processes and react with G. stearothermophilus to generate fluorescent species with improved fluorescence yield, as compared to nonhalogenated and fluorinated analogs, at conditions in a pH range of 5.5 to 9.0.

The chlorinated 4′-alkylumbelliferyl α-D-glucopyranosides are represented by the structural formula

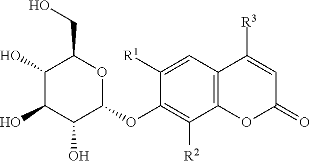

One of R¹ and R² is Cl and the other is H. For example, R¹ may be Cl and R² is H as shown below:

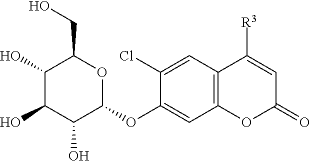

Alternatively, R¹ may be H and R² is Cl as shown below:

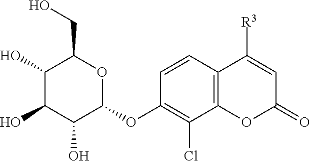

R³ represents an alkyl group having 1 to 12 carbon atoms, preferably 1 to 4 carbon atoms, and more preferably methyl. Examples of R^{b 3} include methyl, ethyl, n-propyl, n-butyl, n-pentyl, isopentyl, n-hexyl, cyclohexyl, n-heptyl, n-octyl, n-nonyl, n-decyl, n-undecyl, and n-dodecyl.

Monochlorinated 4′-alkylumbelliferyl α-D-glucopyranosides according to the above formulas can be prepared by well known general techniques, including, for example, those describes in the examples hereinbelow.

For example, acid-catalyzed cycloaddition of 2-chlororesorcinol or 4-chlororesorcinol with an ethyl (or methyl) 3-oxoalkanoate having the formula

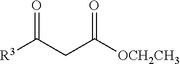

wherein R³ is as previously defined results in corresponding chlorinated 4-alkylumbelliferones. Such ethyl (or methyl) 3-oxoalkanoates can be readily obtained from commercial sources and/or made by conventional methods.

Chlorinated 4-alkylumbelliferones can be coupled to β-D-glucose pentaacetate using boron trifluoride etherate followed by treatment with methanolic methoxide ion to remove acetate groups.

The present disclosure also provides a method of assessing efficacy of a sterilization process, the method comprising sequentially:

a) providing a biological sterilization indicator comprising:
i) bacterial spores comprising, and/or capable of producing, an enzyme capable of catalyzing cleavage of an enzyme substrate represented by the structural formula

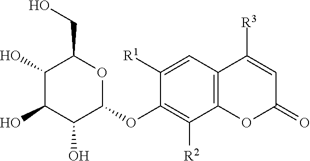

wherein:
   one of R¹ and R² is Cl and the other is H, and
   R³ represents an alkyl group having 1 to 12 carbon atoms; and
ii) a composition, wherein the composition comprises the enzyme substrate, wherein if the composition is brought into contact with the bacterial spores to form a mixture, the mixture will have an initial pH in the range from 5.5 to 9.0.

Next, the bacterial spores are exposed to the sterilization process, and then contacted with the composition ii) above. Viable spores act upon the enzyme substrate thereby cleaving the α-D-glucopyranoside residue and generating the corresponding chlorinated 4-alkylumbelliferone, which is highly fluorescent and may be detected readily by fluorescence, although other method of detection may also be used. Based at least in part on those results, an evaluation is made regarding the efficacy of the sterilization process.

Generally, microorganisms (spores) are chosen to be used as a biological sterilization indicator that are particularly resistant to a given sterilization process. Biological sterilization indicators typically include a viable culture of a known species of microorganism, usually in the form of microbial spores. Bacterial spores, rather than the vegetative form of the organisms, are used at least partly because vegetative bacteria are known to be relatively easily killed by sterilizing processes. Additionally, spores also have superior storage characteristics and could remain in their dormant state for years. As a result, sterilization of an inoculum of a standardized spore strain provides a higher degree of confidence that inactivation of all microorganisms in a sterilizing chamber has occurred.

By way of example only, the present disclosure describes the microorganisms used in the biological sterilization indicator as being “spores;” however, it should be understood that the type of microorganism (e.g., spore) used is typically selected for being resistant to the particular sterilization process contemplated (more resistant than the microorganisms normally present on the items to be sterilized so that inactivation of the test microorganisms indicates a successful sterilization).

In general, the spores used in a particular system are selected according to the sterilization process at hand. For example, for a steam sterilization process, Geobacillus stearothermophilus (G. stearothermophilus) can be used. While demonstrated for G. stearothermophilus, the compounds and methods of the present disclosure may also be used with other biological sterilization indicators, for example, if as they produce appropriate enzymes to cleave the α-D-glucopyranoside moiety from the chlorinated 4-alkylumbelliferyl moiety.

The bacterial spores either comprise an enzyme capable of catalyzing the cleavage of a monochlorinated 4′-alkylumbelliferyl α-D-glucopyranoside according to Formula I to produce a fluorescently-detectable compound, or are capable of producing such an enzyme, or both. The enzymes useful in biological sterilization indicators of the present disclosure include extracellular and intracellular enzymes whose activity correlates with the viability of at least one of the microorganisms commonly used to monitor sterilization efficacy (“test microorganism” or “test spores”). In this context, “correlates” means that the enzyme activity, over background, can be used to predict growth of the test microorganism. The enzyme should be one which, following a sterilization cycle which is sublethal to the test microorganism, remains sufficiently active to react with a substrate for the enzyme, within twenty-four hours, and in preferred embodiment within eight hours or less, yet be inactivated or appreciably reduced in activity following a sterilization cycle which would be lethal to the test microorganism.

When acted upon by an enzyme (e.g., α-glucosidase) produced by G. stearothermophilus, chlorinated 4′-alkylumbelliferyl α-D-glucopyranosides according to the present disclosure are cleaved to form the fluorescent compounds shown below:

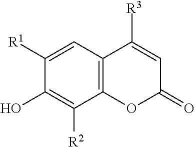

which is in equilibrium with the deprotonated forms (shown below):

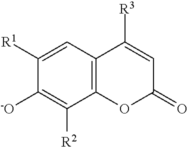

in relative amounts depending on pH.

Methods of assessing sterilization efficacy according to the present disclosure may be used to monitor the effectiveness of one or more types of sterilization procedures, including sterilization procedures that use various sterilants such as steam (e.g., pressurized steam), vapor phase hydrogen peroxide (which may or may not include hydrogen peroxide plasma), ethylene oxide gas, dry heat, propylene oxide gas, methyl bromide, chlorine dioxide, formaldehyde and peracetic acid (alone or with a vapor phase of another material), ozone, radiation, and combinations thereof.

In at least some of the sterilization processes, an elevated temperature, for example, 50° C., 60° C., 100° C., 121° C., 132° C., 134° C., or 135° C., is included or may be encountered in the process. In addition, elevated pressures and/or a vacuum may be encountered, for example, 15 psi (0.10 MPa) at different stages within a single given sterilization cycle, or in different sterilization cycles.

In the case of steam being the sterilant, the sterilization temperatures can include 121° C., 132° C., 134° C., or 135° C. The instant biological sterilization indicators are suitable for steam sterilization cycles at each of the temperatures above and for each temperature the cycle can have a different air removal process chosen from gravity, pre-vacuum (“pre-vac”), and steam flush pressure pulse (SFPP). Each of these cycles may have different exposure times depending on the type of instruments/devices being sterilized. In this disclosure, pre-vacuum and SFPP are also labeled as Dynamic Air Removal (DAR) cycles.

A tabular representation of exemplary steam sterilization cycles in which the present biological sterilization indicators can be used is shown below:

| 121° C. | | | 132° C. | | | 134° C. | | | 135° C. | | |
|---|---|---|---|---|---|---|---|---|---|---|---|
| Gravity | Pre-Vac | SFPP | Gravity | Pre-Vac | SFPP | Gravity | Pre-Vac | SFPP | Gravity | Pre-Vac | SFPP |

In this disclosure, the term a “T gravity” sterilization cycle refers to a steam process where the sterilization temperature is T and air is removed (conditioning) from the sterilization chamber as a result of steam displacement. In this case, the force of gravity causes the heavier gas (air) to exit the chamber via the sterilizer drain as steam enters the chamber. In general, gravity cycles require more exposure time because the air removal method is more passive in nature. For instance, a “121 gravity” cycle is a steam sterilization carried out at 121° C. under gravity conditioning.

A “T pre-vac” sterilization cycle refers to a steam process where the sterilization temperature is T and where air removal is done by mechanical vacuum evacuation in conjunction with steam injections. As a consequence of this conditioning method, the pressure in the sterilization chamber can decrease below atmospheric values during the evacuation cycle and can increase to positive pressures when steam is being introduced. For instance, “121 pre-vac” sterilization cycle refers to a steam process where the sterilization temperature is 121° C. and the conditioning occurs via vacuum evacuations.

A “T SFPP” sterilization cycle refers to a steam process where the sterilization temperature is T° C. and where conditioning is carried out through a series of pressurizations and flushes with steam. During a SFPP process, the pressure in the chamber does not drop below atmospheric (no vacuum is drawn). For example, a “121 SFPP cycle refers to a steam process where the sterilization temperature is 121° C. and the conditioning occurs via steam flush pressure pulses.

In this disclosure, a “dynamic air removal” cycle refers to a sterilization cycle that uses either pre-vacuum or SFPP conditioning.

In other embodiments, methods accord to the present disclosure may be used to monitor the effectiveness of a vapor phase sterilization procedure that uses an oxidizing sterilant. In some embodiments, the methods may be used to monitor the effectiveness of any of the hydrogen peroxide sterilization procedures known in the art. More preferably, the methods may be used to monitor the effectiveness of a hydrogen peroxide vapor phase sterilization procedure.

While aqueous hydrogen peroxide (H₂O₂) has a long history of use as a sterilant, the concept of vapor-phase hydrogen peroxide (VPHP) sterilization has been developed within the past decade. This process is a low temperature sterilization process that kills a wide range of microorganisms including bacterial endospore-forming bacteria commonly used as challenge organisms to evaluate and validate the effectiveness of sterilization cycles in hospitals. A major advantage of hydrogen peroxide is its short exposure cycle time (few minutes). Furthermore, at the end of a hydrogen peroxide sterilization process. only air and water remain in the chamber.

In general, a sterilization process includes placing the selected bacterial spores in a sterilizer. In some embodiments, the sterilizer includes a sterilization chamber that can be sized to accommodate a plurality of articles to be sterilized, and can be equipped with a means of evacuating air and/or other gases from the chamber and a means for adding a sterilant to the chamber. The bacterial spores, typically in a open container, can be positioned in areas of the sterilizer that are most difficult to sterilize. Alternately, the bacterial spores can be positioned in process challenge devices to simulate sterilization conditions where sterilant may not be delivered as directly as would be the case in more favorable sterilization circumstances.

The sterilant can be added to the sterilization chamber after evacuating the chamber of at least a portion of any air or other gas present in the chamber. Alternatively, sterilant can be added to the chamber without evacuating the chamber. A series of evacuation steps can be used to assure that the sterilant reaches all desired areas within the chamber and contacts all desired article(s) to be sterilized, including the bacterial spores.

Methods according to the present disclosure are preferably capable of determining the efficacy of one or more steam sterilization cycles chosen from the powerset of the following eleven cycles: 121 gravity, 121 pre-vac, 121 SFPP, 132 gravity, 132 pre-vac, 132 SFPP, 134 pre-vac, 134 SFPP, 135 gravity, 135 pre-vac, and 135 SFPP, preferably within 1 hr.

The composition used in methods according to the present disclosure contains one or more enzyme substrates according to the present disclosure. In some embodiments, the composition may also include nutrients for the spores, such as germination nutrients that allow germination and/or growth of any viable surviving spores. In some embodiments, the composition is solid (e.g., a powder).

Suitable nutrients may be provided initially in a dry form (e.g., powdered form, tablet form, caplet form, capsule form, a film or coating, entrapped in a bead or other carrier, another suitable shape or configuration, or a combination thereof) and then optionally combined with a suitable solvent to provide a composition that is then combined with the bacterial spores; for example, in a frangible container.

In some embodiments, the composition is liquid (i.e., a liquid composition). In some of those embodiments, the solvent of the liquid composition is water. The combination of nutrients form a nutrient medium and together with the enzyme substrate and one or more non-nutrient components such as indicators, buffer components, salts, solvent, etc. (see below) form the composition.

The nutrients in the composition can include one or more sugars, including, for example, glucose, fructose, dextrose, maltose, trehalose, cellobiose, or the like, or a combination thereof. Alternatively, the nutrients may include complex media such as, for example, peptone, tryptone, phytone peptone, yeast extract, soybean casein digest, other extracts, hydrolysates, or a combination thereof. In other embodiments, the nutrients in the composition represent a combination of one or more complex media components and other specific nutrients. The nutrient medium can also include a salt, including, but not limited to, sodium chloride, potassium chloride, calcium chloride, or the like, or a combination thereof. In some embodiments, the nutrient can further include at least one amino acid, including, but not limited to, at least one of methionine, phenylalanine, alanine, tyrosine, and tryptophan.

If part of a self-contained biological sterilization indicator, the composition comprising nutrients, the enzyme substrate, and other components is typically present throughout the sterilization procedure but may be kept separate and not accessible to the sources of biological activity in a frangible container until desired. After the sterilization process is completed and the biological sterilization indicator is used to determine the efficacy of the sterilization, the composition is placed in contact with the spores resulting in a mixture. In this disclosure, placing the composition with the spores includes rupturing the frangible container (e.g., by fracturing, puncturing, piercing, crushing, cracking, or breaking) so that the composition is released and contacts the spores. This process may include mixing of the composition with the spores, such as by manual or mechanical shaking of a housing containing the frangible container and the bacterial spores so that the composition adequately mixes with the spores.

The process of bringing the bacterial spores and composition including a chlorinated 4′-alkylumbelliferyl α-D-glucopyranoside according to the present disclosure causes viable bacterial enzymes to remove the glucopyranoside moiety and generate the fluorescent anionic form of the corresponding chlorinated 4-umbelliferone.

In some embodiments, the mixture resulting from mixing the composition with the spores after activation remains isolated within a housing of a biological sterilization indicator after the sterilization cycle has been completed and no additional reagents or components are added to it during or after activation. If the spores are viable and grow, then the enzyme produced by the bacteria catalyzes the cleavage of the enzyme substrate, which produces the fluorescently-detectable compound. This means that the same solution in the same container (e.g., a housing) is used for three separate events: (a) spore germination/growth, if the spores are viable, (b) the enzymatic cleavage of the enzyme substrate, resulting the production of the fluorescently-detectable compound, and (c) the fluorescence detection of the fluorescently-detectable compound.

In some embodiments, the composition may comprise a buffered solution. The ionic conditions of the buffered solution should be such that the enzyme and enzyme substrate are not affected. In some embodiments, a buffer solution is used as part of the composition, such as phosphate buffers, (e.g., phosphate buffered saline solution, potassium phosphate or potassium phosphate dibasic), tris(hydroxymethyl) aminomethane-HCl solution, or acetate buffer, or any other buffer suitable for sterilization known in the art. Buffers suitable for the present biological sterilization indicators should be compatible with fluorogenic and chromogenic enzyme substrates used as part of the composition. Another consideration in choosing the buffers is their influence on the enzyme activity. For example, phosphate buffered saline contains a relatively high concentration of inorganic phosphate, which is a competitive inhibitor of alkaline phosphatase. Thus, for that enzyme, a Tris-HCl buffer is recommended. The strength of the buffered solution may be from 0.05 M to 0.5 M, preferably from 0.05 M to 0.25 M, more preferably from 0.05 M to 0.15 M, even more preferably about 0.1 M.

The concentration of enzyme substrate present in the composition depends upon the identity of the particular substrate and enzyme, the amount of enzyme-product that must be generated to be detectable, either visually or by instrument, and the amount of time that one is willing to wait in order to determine whether active enzyme is present in the reaction mixture. Preferably, the amount of enzyme substrate is sufficient to react with any residual active enzyme present, after the sterilization cycle, within about an eight-hour period of time, such that at least 10⁻⁸ molar enzyme-modified product is produced.

In some embodiments, the composition comprises a solution adjusted to a suitable pH, but without an added buffer system. In other embodiments, however, the composition does comprise a buffered solution.

In some embodiments, the methods may further comprise an additional indicator compound that can facilitate the detection of another metabolic activity of the test microorganisms (e.g., spore) (aside from an enzyme substrate that can produce a fluorescently-detectable compound). This additional metabolic activity can also be an enzymatic activity. Non-limiting examples of indicator compounds include a chromogenic enzyme substrate (e.g., observable in the visible spectrum), a pH indicator, a redox indicator, a chemiluminescent enzyme substrate, a dye, and a combination of any two or more of the foregoing indicator compounds.

In some embodiments, the additional indicator is a pH indicator that produces a change in color when the pH decreases, indicating growth of the test microorganisms. In some embodiments, the pH indicator is bromocresol purple. The pH indicator can be used to detect a second biological activity, such as the fermentation of a carbohydrate to acid end products (suggesting survival of the test microorganisms) and an enzymatic biological activity such as α-D-glucosidase enzyme activity, for example. These activities can indicate the presence or absence of a viable spore following the exposure of a biological sterilization indicator to a sterilization process, for example. The bromocresol purple can be used at a concentration of about 0.03 g/L in the aqueous mixture, for example. Enzyme substrates according the present disclosure can be used, for example, at a concentration of about 0.05 to about 0.5 g/L (e.g., about 0.05 g/L, about 0.06 g/L, about 0.07 g/L, about 0.08 g/L, about 0.09 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L) in the aqueous mixture.

In some embodiments, one or more neutralizers, which are not an enzyme and not a metal catalyst may be present during methods according to the present disclosure. A neutralizer is a compound or material that reacts with residual sterilant, e.g., hydrogen peroxide, to neutralize its effect, wherein the neutralizer is not an enzyme, and not a metal catalyst. Enzyme neutralizers are typically not stable at the high temperatures, and thus typically not desirable.

Examples of neutralizers include sulfur containing materials such as methionine, L-cysteine. D-ethionine, S-methyl-L-cysteine, S-benzyl-L-cysteine, sodium thiosulfate, glutathionine, L-cystathionine, N-acetyl-L-cysteine, carboxymethylcysteine, D,L-homocysteine, D,L-homocysteine-thiolactone, and thiodipropionic acid, and non-sulfur containing materials such as isoascorbic acid, potassium ferricyanide, and sodium pyruvate.

### Detection of Enzymatic Activity and Determination of a Successful Sterilization Process

After the sterilization process, the spores can be incubated in a nutrient medium to determine whether any of the spores survived the sterilization process, with spore growth indicating that the sterilization process was insufficient to destroy all of the test microorganisms.

After activation, the mixture resulting from placing the composition in contact with the spores is incubated for a period of time and under conditions that would be sufficient to liberate a detectable amount of the enzyme modified product, assuming, of course, that any of the spores remain active. In general, the amount of product which is detectable by known methods is at least 10⁻⁸ molar. Preferably. the incubation conditions are sufficient to generate at least 10⁻⁸ molar of fluorescently-detectable compound, more preferably, at least about 10⁻⁶ molar or even at least about 10⁻⁵ molar of fluorescently-detectable compound. The incubation time and temperature needed to produce a detectable amount of fluorescently-detectable compound will depend upon the identity of the enzyme and the substrate, and the concentrations of each present in the reaction mixture. In general, the incubation time required is between about 1 minute and 12 hours, and the incubation temperature is between about 20° C. and 70° C. For example, where G. stearothermophilus is the source of the enzyme, the incubation time may be from about 1 minute to 3 hours, or from 1 minute to 1 hour, or from 1 minute to 30 minutes, or from 1 minute to 10 minutes, and the incubation temperature is from about 52° C. to 65° C.

To detect a detectable change in the spores the mixture can be assayed immediately after the composition and the spores have been combined to achieve a baseline reading. After that, any detectable change from the baseline reading can be detected. The mixture can be monitored and measured continuously or intermittently. In some embodiments, a portion of, or the entire, incubating step may be carried out prior to measuring the detectable change. In some embodiments, incubation can be carried out at a first temperature (e.g., at 37° C., or at 50-60° C.), and measuring of the detectable change can be carried out at a second different temperature (e.g., at room temperature, 25° C., or at 37° C.). In other embodiments, the incubation and measurement of fluorescence occurs at the same temperature.

The readout time of the mixture (i.e., the time to determine the effectiveness of the sterilization process) can be, in some embodiments, less than 8 hours, in some embodiments, less than 1 hour, in some embodiments, less than 30 minutes, in some embodiments, less than 15 minutes, in some embodiments, less than 5 minutes, and in some embodiments, less than 1 minute. In other embodiments, the readout time for the biological sterilization indicator of this disclosure is from 2 min to 1 hr, or from 2 min to 50 min, or from 2-30 min, or from 2-20 min, or from 2-25 min, or from 5 to 30 min, or from 5-25 min, or from 5-20 min. The detection of fluorescence above the baseline reading that would indicate presence of viable spores (i.e., a failed sterilization process) can be performed according to any method know in the art, including area under curve (in a plot of time vs fluorescence intensity), monitoring a change in slope of the curve, using a threshold value for the fluorescence, etc., or a combination thereof of two or more techniques.

The above-described process can be conducted in many possible configurations. One such configuration involving a particular sterilization indicator device that contains the composition and the spores is described in PCT Publication WO 2020/115661 A1 (Bonilla et al.), incorporated herein by reference, except that chlorinated 4′-alkylumbelliferyl α-D-glucopyranosides according to the present disclosure is exchanged for the fluorinated 4′-alkylumbelliferyl α-D-glucopyranosides in that publication.

Objects and advantages of this disclosure are further illustrated by the following non-limiting examples, but the particular materials and amounts thereof recited in these examples, as well as other conditions and details, should not be construed to unduly limit this disclosure.

### EXAMPLES

Unless otherwise noted, all parts, percentages, ratios, etc. in the Examples and the rest of the specification are by weight.

Column chromatography purification of compounds was conducted using an ISOLARA HPFC system (an automated high-performance flash chromatography purification instrument available from Biotage, Inc, Charlottesville, Virginia). The eluent used for each purification is described in the examples.

Proton nuclear magnetic resonance (¹H NMR and ¹⁹F NMR) analyses were conducted using a BRUKER A500 NMR spectrometer (Bruker Corporation, Billerica, Massachusetts).

Methanesulfonic acid, □-D-glucose pentaacetate, DMSO and 4-(dimethylamino)pyridine (DMAP) were obtained from Alfa Aesar, Ward Hill, Massachusetts.

Ethyl acetoacetate, 4-chlororesorcinol and 2-chlororesorcinol were obtained from Oakwood Chemical, Estill, South Carolina.

Tryptone (# BD211705) peptone C (BD 211921), phytone peptone (BD BBL 211906) and yeast extract (# BD212750) were obtained from Becton, Dickinson and Company, Franklin Lakes, New Jersey.

Deionized water was purified using a MILLI-Q water purification system (EMD Millipore, Burlington, Massachusetts).

Suspensions of Geobacillus stearothermophilus spores were prepared using molecular biology grade water (# BP2819) obtained from Thermo Fisher Scientific, Waltham, Massachusetts. 4′-Methylumbelliferyl-□-D-glucopyranoside (MUG) was obtained from Biosynth AG, Switzerland.

Boron trifluoride etherate obtained from the Sigma-Aldrich Company, St. Louis, MO.

Culture media was prepared by dissolving by dissolving 2 g/L phytone peptone (BD BBL 211906) and 0.2 g/L L-alanine (Sigma 05129) in deionized water. 1N NaOH or 0.1 N HCl were used to adjust pH, and media were brought to volume and filter sterilizing using a 0.22 micron polyethersulfone (PES) filter. Stock solutions (25 mg/ml) of 6′-chloro-4′-methylumbelliferyl-a-D-glucopyranoside, 8′-chloro-4′-methylumbelliferyl-a-D-glucopyranoside, 8′-fluoro-4′-methylumbelliferyl-a-D-glucopyranoside, and 4-methylumbelliferyl-a-D-glucopyranoside were prepared in DMSO. The fluorogenic a-glucosidase substrates were added to culture media at a final concentration of 0.3 mg/ml.

### Comparative Example A

### Preparation of 8′-fluoro-4′-methylumbelliferyl α-D-glucopyranoside (8F-MUG, Enzyme Substrate)

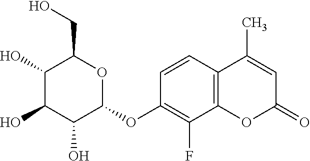 [SMILES: Cc1cc(=O)oc2c(F)c(O[C@H]3O[C@H](CO)[C@@H](O)[C@H](O)[C@H]3O)ccc12]

Part A: 2,3,4-Trifluoronitrobenzene (27.01 g, 0.153 mol) was dissolved in methanol (200 mL) under a nitrogen atmosphere. The stirred solution was cooled in an ice bath. A 5 M solution of sodium methoxide in methanol (70 mL) was slowly added over a period of 3 hours. The resulting mixture was left to warm to ambient temperature overnight. The reaction was then quenched with 60 mL of a 1 M aqueous citric acid solution and the solvent was removed under reduced pressure. Diethyl ether (400 mL) and an additional 100 mL of 1 M citric acid solution were added. The layers were separated and the organic portion was washed successively with 100 mL of 1 M citric acid solution, deionized water and brine. The organic portion was dried over magnesium sulfate, filtered, and concentrated under reduced pressure to give 28.4 g of 2,4-dimethoxy-3-fluoronitrobenzene as a brown solid. ¹⁹F NMR (470 MHZ, CDCl₃) δ−148.9 (d, J=7.4 Hz), ¹H NMR (500 MHZ, CDCl₃) δ 7.74 (dd, J=9.4, 2.2 Hz, 1H), 6.74 (dd, J=9.3, 7.5 Hz, 1H), 4.07 (d, J=1.8 Hz, 3H), 3.97 (s, 3H).

Part B: 2,4-Dimethoxy-3-fluoronitrobenzene (28.0 g, 0.139 mol) was dissolved in 50 mL of ethyl acetate and the mixture was transferred to a 500 mL Parr hydrogenation bottle. Ethanol (50 mL) was added and the solution degassed for 20 minutes with a stream of nitrogen. Palladium (10%) on carbon (1.25 g) was added and the mixture was shaken under an atmosphere of hydrogen (45 PSI). When hydrogen consumption had ceased, the reaction mixture was filtered through a CELITE pad to yield a pale pink solution. The solvent was removed under reduced pressure to give 22.9 g of 1-amino-3-fluoro -2,4-dimethoxybenzene as an orange oil. ¹⁹F NMR (470 MHZ, CDCl₃) δ−152.6 (d, J=8.3 Hz). ¹H NMR (500 MHz, CDCl₃) δ 6.54 (t, J=8.7 Hz, 1H), 6.42 (dd, J=8.2, 2.2 Hz, 1H), 3.93 (d, J=1.5 Hz, 3H), 3.81 (s, 3H), 3.4 (broad s, 2H).

Part C: A stirred suspension of 1-amino-3-fluoro-2,4-dimethoxybenzene (4.8 g, 0.027 mol) in a 2:1 mixture of water and concentrated hydrochloric acid (90 mL) was cooled to −5° C. A pre-cooled solution (4° C.) of sodium nitrite (2.05 g, 0.030 mol) dissolved in 6 mL of deionized water was added and the mixture was stirred for 15 minutes, during which more sodium nitrite (0.5 g=7.3 mmol in 1 mL deionized water) was added. A cooled (10° C.) hypophosphorous acid solution (57 mL, 50% w/w in water, Alfa Aesar) was added in several portions over 6-7 minutes, and the reaction mixture left in the refrigerator overnight. The following morning it was warmed to room temperature with stirring, diluted with 350 mL deionized water, followed by enough 1N NaOH to neutralize the solution. The mixture was extracted with diethyl ether (2×500 mL) and the combined organic phases were extracted with water (2×) and brine, then dried with magnesium sulfate and filtered. The filtrate was concentrated under reduced pressure to yield a red oil. Column chromatography (0.5%-10% ethyl acetate/hexanes) provided 1.8 g of 2-fluoro-1,3-dimethoxybenzene as a pale orange liquid. ¹⁹F NMR (470 MHz, CDCl₃) δ−159.00 (t, J=7.0 Hz). ¹H NMR (500 MHZ, CDCl₃) δ 6.98 (td, J=8.4, 2.3 Hz, 1H), 6.61 (dd, J=8.4 Hz, 7.4 Hz, 2H), 3.88 (s, 6H).

Part D: 2-Fluoro-1,3-dimethoxybenzene (1.78 g, 11.4 mmol) was dissolved in anhydrous dichloromethane (10 mL) and a 1.0 M solution of BBr₃ in dry dichloromethane (34 mL) was added through a dropping funnel over 30 minutes. The reaction mixture was stirred for two days, poured into 350 mL of deionized water and then stirred vigorously for 3 hours. The aqueous mixture was extracted with diethyl ether (2×150 mL), and then the combined organic fractions were washed with deionized water and brine, dried over magnesium sulfate, filtered, and concentrated under reduced pressure to yield 1.09 g of 2-fluororesorcinol as a tan solid. ¹⁹F NMR (470 MHz, acetone-d₆) δ−163.6 (t, J=7.3 Hz). ¹H NMR (500 MHZ, acetone-d₆) δ 8.48 (s, 2H), 6.77 (td, J=8.2, 1.9 Hz, 1H), 6.46 (t, J=7.9 Hz, 2H).

Part E: 2-Fluororesorcinol (4.00 g) was dissolved in ethyl acetoacetate (4.5 mL) in an ice bath. Methanesulfonic acid (40 mL) was added to the solution dropwise over a period of about 30 minutes. The ice bath was removed, and the reaction was stirred overnight. The solution was then poured into deionized water. The resulting precipitate was recovered by filtration and then air dried to provide 6.07 g of product. Column chromatography (5%-20% acetone/chloroform) yielded 8-fluoro-4-methylumbelliferone as a white solid. ¹⁹F NMR (470 MHz, acetone-d₆) □−160.81 (dd, J=7.7, 1.1 Hz). ¹H NMR (500 MHZ, acetone-d₆) 9.63 (s, 1H), 7.43 (dd, J=8.9, 2.0 Hz, 1H), 6.99 (dd, J=8.8, 7.8 Hz, 1H), 6.16 (d, J=1.2 Hz, 1H), 2.30 (d, J=1.2 Hz, 3H).

Part F: A 500-mL round bottom flask was charged with β-D-glucose pentaacetate (2.01 g, 5.15 mmol, Alfa Aesar), DMAP (1.26 g, 10.3 mmol, Alfa Aesar) and 8-fluoro-4-methylumbelliferone (1.00 g, 5.15 mmol). Anhydrous 1,2-dichloroethane (20 mL) was added and the mixture was stirred under an atmosphere of nitrogen. Boron trifluoride etherate (4.13 mL, 33.5 mmol, Sigma-Aldrich Co.) was then carefully added to the stirred reaction mixture which soon became homogenous. The reaction mixture was then heated to 60° C. After 3 h, the reaction mixture was cooled, diluted with CHCl₃ and carefully quenched by addition of saturated sodium bicarbonate solution. The reaction mixture was placed in a separatory funnel and the layers were separated. The organic portion was washed with of 1N NaOH solution, followed by washing with water and brine. The organic portion was dried over Na₂SO₄, filtered and concentrated. Column chromatography (SiO₂, 50-75% ethyl acetate/hexanes) gave 4′-methyl-8′-fluoroumbelliferyl 2,3,4,6-tetra-O-acetyl-D-glucopyranoside as a mixture of the alpha and beta anomers. These isomers were separated by running a second silica column chromatography (SiO₂, 40-60% ethyl acetate/hexanes) to give 1.25 g of purified 1-O-8′-fluoro-4′-methyl umbelliferyl-2,3,4,6-tetra-O-acetyl-α-D-glucose as a white powder. ¹H NMR (500 MHZ, CDCl₃) δ 7.31 (dd, J=2.0, 9.0 Hz, 1H), 7.10 (dd, J=6.8. 8.9 Hz, 1H), 6.27 (d, J=1.2 Hz, 1H), 5.79 (d, J=3.8 Hz, 1H), 5.72 (t, J=9.8 Hz, 1H), 5.19 (t, J=9.8 Hz, 1H), 5.03 (dd, J=3.7, 10.3 Hz, 1H), 4.23-4.31 (m, 2H), 4.06-4.16 (m, 1H), 2.43 (d, J=1.2 Hz, 3H), 2.12 (s, 3H), 2.05-2.08 (m, 9H).

Part G: 8′-fluoro-4′-methylumbelliferyl 2,3,4,6-tetra-O-acetyl-α-D-glucopyranoside (0.78 g) was dissolved in 20 mL of anhydrous methanol and treated with 5 drops of 5 M sodium methoxide solution (TCI, Tokyo, Japan). After stirring for 2 hours, a white precipitate formed. The solid was isolated by filtration, rinsed with a small amount of cold methanol and dried with suction to give 345 mg of white powder. Crystallization from methanol gave purified 8′-fluoro-4′-methylumbelliferyl α-D -glucopyranoside as a white powder. ¹H NMR (500 MHZ, methanol-d₄) δ 7.54 (dd, J=1.5, 9.0 Hz, 1H), 7.40 (dd, J=7.3, 8.9 Hz, 1H), 6.29 (s, 1H), 5.73 (d, J=3.5 Hz, 1H), 3.93 (t, J=9.3 Hz, 1H), 3.68-3.78 (m, 3H), 3.66 (dd, J=3.5, 9.8 Hz, 1H), 3.43-3.50 (m, 1H), 2.48 (s, 3H)

### Preparative Example 1

### Preparation of 6-chloro-4-methylumbelliferone

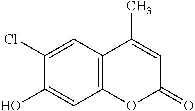

4-Chlororesorcinol (2.01 g, 13.8 mmol) was stirred in ethyl acetoacetate (2.2 mL, 17.2 mmol) in an ice bath. Methanesulfonic acid (20 mL) was added to the solution dropwise over a period of about 30 minutes, and the reaction mixture allowed to warm up overnight with stirring. The solution was then added dropwise to vigorously stirred deionized water (500 mL), and stirred for a further 120 minutes. The solution was then filtered and the solid dried under reduced pressure. The product was then recrystallized from hot methanol. ¹H NMR (500 MHZ, acetone-d₆) δ 11.4 (broad, 1H), 7.76 (s, 1H), 6.90 (s, 1H), 6.21 (d, J=1.2 Hz, 1H), 2.38 (d, J=1.2 Hz, 3H). ¹³C NMR (126 MHZ, DMSO-d₆) δ 160.3, 156.7, 153.5, 153.4, 126.6, 117.3, 113.3, 111.8, 103.8, 18.6.

### Preparative Example 2

### Preparation of 8-chloro-4-methylumbelliferone

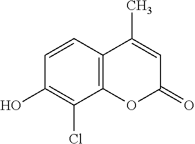

2-Chlororesorcinol (5.01 g, 34.6 mmol) was dissolved in ethyl acetoacetate (5.0 mL, 39.2 mmol) and cooled in an ice bath. Methanesulfonic acid (40 mL) was added to the solution dropwise over a period of about 30 minutes, and the reaction mixture allowed to warm up overnight with stirring. The solution was then added dropwise to vigorously stirred deionized water (1L), and stirred for a further 30 minutes. The solution was then filtered and the solid dried under reduced pressure. Recrystallized from hot methanol as fine needles. ¹H NMR(500 MHZ, acetone-d₆) δ 9.74 (broad, 1H), 7.72 (d, J=8.8 Hz, 1H), 7.04 (d, J=8.8 Hz, 1H), 6.18 (q, J=1.2 Hz, 1H), 2.38 (d, J=1.2 Hz, 3H). ¹³C NMR (126 MHZ, acetone-d₆) δ 159.5, 156.8, 153.4, 151.4, 124.4, 114.2, 112.7, 111.7, 107.8, 18.1.

### Example 1

### Preparation of 6′-chloro-4′-methylumbelliferyl α-D-glucopyranoside

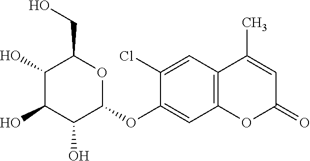 [SMILES: Cc1cc(=O)oc2cc(O[C@H]3O[C@H](CO)[C@@H](O)[C@H](O)[C@H]3O)c(Cl)cc12]

Part A: A 500-mL round bottom flask was charged with β-D-glucose pentaacetate (3.61 g, 9.28 mmol), DMAP (4.53 g, 37.1 mmol) and 6-chloro-4-methylumbelliferone (1.95 g, 9.28 mmol). Anhydrous 1,2-dichloroethane (80 mL) was added and the mixture was stirred under an atmosphere of nitrogen. Boron trifluoride etherate (14.3 mL, 116 mmol) was then carefully added to the stirred reaction mixture and the solution soon became homogenous. The reaction mixture was then heated to 60° C. After 4 h, the reaction mixture was cooled and carefully quenched by addition of saturated sodium bicarbonate solution. The reaction mixture was diluted with 100 mL of ethyl acetate filtered through a pad of Celite to remove insoluble material. The filtrate was placed in a separatory funnel and the layers were separated. The organic portion was washed sequentially with 0.5N NaOH solution, water, and brine. The organic portion was dried over Na₂SO₄, filtered and concentrated. Column chromatography (silica gel eluting with 5-7.5% acetone/CHCl₃) gave a partially purified mixture of glucosides, with unidentified impurities, as a white solid. The solid was dissolved in 15 mL of hot ethyl acetate and cooled. The resulting solid was removed by filtration and the filtrate (containing mostly glucosides) was concentrated. A second column (silica gel eluting with 25-50% ethyl acetate/hexanes) gave 0.55 g of purified 6′-chloro -4′-methyl umbelliferyl-2,3,4,6-tetraacetyl glucose as a 3:1 mixture of □□□ anomers. The mixture of anomers was separated by column chromatography (silica gel eluting with 2%-10% acetone/CHCl₃) to give 146 mg of 6′-chloro-4′-methylumbelliferyl 2,3,4,6-tetra-O-acetyl-α-D-glucopyranoside as a white solid. ¹H NMR(500 MHZ, CDCl₃) δ ppm 7.62 (s, 1H), 7.23 (s, 1H), 6.24 (d, J=1.1 Hz, 1H), 5.81 (d,

J=3.7 Hz, 1H), 5.73 (t, J=9.8 Hz, 1H), 5.17 (t, J=9.8 Hz, 1H), 5.05 (dd, J=10.3, 3.7 Hz, 1H), 4.29 (dd, J=12.3, 5.1 Hz, 1H), 4.17 (ddd, J=10.3, 5.1, 2.0 Hz, 1H), 4.07 (dd, J=12.3, 2.1 Hz, 1H), 2.41 (d, J=1.1 Hz, 3H), 2.10 (s, 3H), 2.09 (s, 3H), 2.07 (s, 3H), 2.06 (s, 3 H).

Part B: 6′-Chloro-4′-methylumbelliferyl 2,3,4,6-tetra-O-acetyl-α-D-glucopy ranoside (146 mg) was dissolved in 5 mL of anhydrous methanol and treated with 2 drops of 5M sodium methoxide solution. After stirring for 60 min, the reaction mixture was concentrated under reduced pressure to give a white solid. The white solid was triturated in 5 mL of hot ethanol, cooled, and filtered to give 54 mg of a white solid. Crystallization from methanol gave 33 mg of 6′-chloro-4′-methylumbelliferyl-□-D- glucopyranoside as white needles. ¹H NMR (500 MHZ, methanol-d₄) δ ppm 7.83 (s, 1H), 7.38 (s, 1 H), 6.27 (s, 1H), 5.80 (d, J=3.4 Hz, 1H), 3.98 (t, J=9.4 Hz, 1H), 3.75 (dd, J=12.1, 2.4 Hz, 1H), 3.67-3.72 (m, 2H), 3.63 (ddd, J=9.9, 4.9, 2.3 Hz, 1H), 3.47 (t, J=9.4 Hz, 1H), 2.46 (d, J=0.9 Hz, 3H).

### Example 2

### Synthesis of 8′-chloro-4′-methylumbelliferyl □-D-glucopyranoside

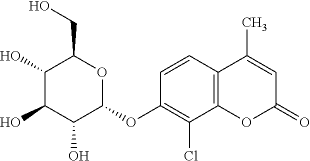 [SMILES: Cc1cc(=O)oc2c(Cl)c(O[C@H]3O[C@H](CO)[C@@H](O)[C@H](O)[C@H]3O)ccc12]

Part A: 500-mL round bottom flask was charged with □-D-glucose pentaacetate (3.61 g, 9.28 mmol), DMAP (1.95 g, 18.6 mmol) and 8-chloro-4-methylumbelliferone (1.95 g, 9.28 mmol). Anhydrous 1,2-dichloroethane (80 mL) was added and the mixture was stirred under an atmosphere of nitrogen. Boron trifluoride etherate (7.2 mL, 58.4 mmol) was then carefully added to the stirred reaction mixture which soon became homogenous. The reaction mixture was then heated to 60° C. After 20 h, the reaction mixture was cooled and carefully quenched by addition of saturated sodium bicarbonate solution. The reaction mixture was diluted with 80 mL of CH₂Cl₂ and the mixture was filtered through a pad of Celite to remove insoluble material. The filtrate was placed in a separatory funnel and the layers were separated. The organic portion was washed with of 0.2 N NaOH solution, followed by washing with water and brine. The organic portion was dried over Na₂SO₄, filtered and concentrated. Column chromatography (silica gel eluting with 5-7.5% acetone/CHCl₃) gave a partially purified mixture of glucosides, with unidentified impurities, as a white solid. The solid was dissolved in 15 mL of hot ethyl acetate and cooled. The resulting solid was removed by filtration and the filtrate (containing mostly glucosides) was concentrated. A second column (silica gel eluting with 25-75% ethyl acetate/hexanes) gave predominately the desired product as a white solid. The white solid was crystallized from methanol to give 562 mg of 8′-chloro-4′-methylumbelliferyl 2,3,4,6-tetra-O-acetyl-□-D-glucopyranoside as white needles. ¹H NMR (500 MHZ, CDCl₃) δ ppm 7.47 (d, J=8.9 Hz, 1H), 7.16 (d, J=8.9 Hz, 1H), 6.26 (d, J=1.1 Hz, 1H), 5.89 (d, J=3.7 Hz, 1H), 5.76 (t, J=9.8 Hz, 1H), 5.21 (t, J=9.8 Hz, 1H), 5.04 (dd, J=10.3, 3.7 Hz, 1H), 4.27 (dd, J=12.4, 4.2, 1H), 4.20 (ddd, J=10.3, 4.1, 2.2 Hz, 1H), 4.09 (dd, J=12.5, 2.2 Hz, 1H), 2.43 (d, J=1.2 Hz, 3H), 2.10 (s, 3H), 2.08 (s, 6H), 2.07 (s, 3H).

Part B: 8′-Chloro-4′-methylumbelliferyl 2,3,4,6-tetra-O-acetyl-□-D-glucopyranoside (562 mg) was dissolved in 10 mL of anhydrous methanol and treated with 2 drops of 5 M sodium methoxide solution. After stirring for 4 hours, a white precipitate formed. The solid was isolated by filtration, rinsed with a small amount of cold methanol to give a pasty white solid. The material was triturated with 10 mL of refluxing methanol, cooled and filtered to give 345 mg of 8′-chloro-4′-methylumbelliferyl α-D-glucopyranoside as a white powder. ¹H NMR (500 MHZ, DMSO-d₆) δ ppm 7.72 (d, J=9.0 Hz, 1H), 7.37 (d, J=9.0 Hz, 1H), 6.34 (s, 1H), 5.80 (d, J=3.3 Hz, 1H), 5.31 (d, J=5.3 Hz, 1H), 5.13 (dd, J=8.2, 5.3 Hz, 2H), 4.49 (m, 1H), 3.74 (td, J=9.2, 5.1 Hz, 1H), 3.42-3.57 (m, 4H), 3.24 (m, 1H), 2.43 (s, 3H). ¹³C NMR (126 MHz, DMSO-d₆) δ ppm 159.65, 155.60, 153.92, 150.31, 124.77, 115.40, 112.58, 112.17, 109.87, 98.42, 75.10, 73.13, 71.75, 69.96, 60.94, 18.70.

### Example 3

Culture media (pH 6.7) containing 6′-chloro-4′-methylumbelliferyl α-D-glucopyranoside (0.3 mg/mL) was each added to eight wells (100 microliters per well) of a black 96-well microtiter plate with (NUNC #165301, Thermo Fisher Scientific, Waltham, Massachusetts). A 5-microliter aqueous suspension containing 5×10³ Geobacillus stearothermophilus spores (ATCC 7953) was added to 4 of the wells for each medium in the microtiter plate. Two control wells were also prepared that did not contain any Geobacillus stearothermophilus spores. The top of the plate was covered with an optically clear sealing film (product #4311971, Applied Biosystems Corporation, Foster City, California). The plate was warmed at 60° C. and shaken in a linear direction. Fluorescence readings (360 nm excitation/450 nm emission at 50% gain) were taken from the bottom of the plate. A reading was taken at an initial time point of 10 seconds and then at +1-minute intervals thereafter. A Synergy Neo2 fluorescence plate reader (BioTek Company, Winooski, Vermont) was used. In Table 1, the mean fluorescence values detected (RFU) at times of 10 seconds, +5 minutes, +10 minutes, and +30 minutes are reported. The mean fluorescence values detected for the control wells were 511 RFU at 10 seconds; 426 RFU at +5 minutes: 373 RFU at +10 minutes, and 310 RFU at +30 minutes.

### Example 4

The procedure of Example 3 was followed, with the exception that the culture media (pH 6.7) containing 6′-chloro-4′-methylumbelliferyl α-D-glucopyranoside (0.3 mg/mL) was replaced with culture media (pH 6.7) containing 8′-chloro-4′-methylumbelliferyl α-D-glucopyranoside (0.3 mg/mL). In Table 1, the mean fluorescence values detected (RFU) at times of 10 seconds, +5 minutes, +10 minutes, and +30 minutes are reported. The mean fluorescence values detected for the control wells were 428 RFU at 10 seconds; 365 RFU at +5 minutes: 330 RFU at +10 minutes, and 292 RFU at +30 minutes.

### Comparative Example B

The procedure of Example 3 was followed, with the exception that the culture media (pH 6.7) containing 6′-chloro-4′-methylumbelliferyl α-D-glucopyranoside (0.3 mg/mL) was replaced with culture media (pH 6.7) containing 4′-methylumbelliferyl α-D-glucopyranoside (0.3 mg/mL) (MUG). In Table 1, the mean fluorescence values detected (RFU) at times of 10 seconds, +5 minutes, +10 minutes, and +30 minutes are reported. The mean fluorescence values detected for the control wells were 402 RFU at 10 seconds; 335 RFU at +5 minutes: 297 RFU at +10 minutes, and 243 RFU at +30 minutes.

### Comparative Example C

The procedure of Example 3 was followed, with the exception that the culture media (pH 6.7) containing 6′-chloro-4′-methylumbelliferyl α-D-glucopyranoside (0.3 mg/mL) was replaced with culture media (pH 6.7) containing 8′-fluoro-4′-methylumbelliferyl α-D-glucopyranoside (0.3 mg/mL) (8F-MUG). In Table 1, the mean fluorescence values detected (RFU) at times of 10 seconds, +5 minutes, +10 minutes, and +30 minutes are reported. The mean fluorescence values detected for the control wells were 354 RFU at 10 seconds; 299 RFU at +5 minutes: 265 RFU at +10 minutes, and 224 RFU at +30 minutes.

TABLE 1

| | | | FLUORESCENCE RESULTS | | | |
|---|---|---|---|---|---|---|
| | | | WITH 5 × 10³ SPORES | | | |
| | pH of | ENZYME | MEASURED FLUORESCENCE, RFU | | | |
| | CULTURE | SUBSTRATE | 10 | +5 | +10 | +30 |
| EXAMPLE | MEDIA | USED | Sec | min | min | min |
| Example 3 | 6.7 | Example 1 | 1013 | 2251 | 4622 | 12582 |
| Example 4 | 6.7 | Example 2 | 1164 | 3013 | 6278 | 15253 |
| Comparative | 6.7 | MUG | 430 | 403 | 464 | 991 |
| Example B | | | | | | |
| Comparative | 6.7 | 8F-MUG | 693 | 1422 | 2869 | 8410 |
| Example C | | | | | | |

### Example 5

Culture media (pH 7.0) containing 6′-chloro-4′-methylumbelliferyl α-D-glucopyranoside (0.3 mg/mL) was each added to eight wells (100 microliters per well) of a black 96-well microtiter plate with (NUNC #165301, Thermo Fisher Scientific, Waltham, Massachusetts). A 5-microliter aqueous suspension containing 5×10³ Geobacillus stearothermophilus spores (ATCC 7953) was added to 4 of the wells for each medium in the microtiter plate. Two control wells were also prepared that did not contain any Geobacillus stearothermophilus spores. The top of the plate was covered with an optically clear sealing film (product #4311971, Applied Biosystems Corporation, Foster City, California). The plate was warmed at 60° C. and shaken in a linear direction. Fluorescence readings (360 nm excitation/450 nm emission at 50% gain) were taken from the bottom of the plate. A reading was taken at an initial time point of 10 seconds and then at +1-minute intervals thereafter. A Synergy Neo2 fluorescence plate reader (BioTek Company, Winooski, Vermont) was used. In Table 2, the mean fluorescence values detected (RFU) at times of 10 seconds, +5 minutes, +10 minutes, and +30 minutes are reported. The mean fluorescence values detected for the control wells were 512 RFU at 10 seconds; 417 RFU at +5 minutes; 363 RFU at +10 minutes, and 308 RFU at +30 minutes.

### Example 6

The procedure of Example 5 was followed, with the exception that the culture media (pH 7.0) containing 6′-chloro-4′-methylumbelliferyl α-D-glucopyranoside (0.3 mg/mL) was replaced with culture media (pH 7.0) containing 8′-chloro-4′-methylumbelliferyl α-D-glucopyranoside (0.3 mg/mL). In Table 2, the mean fluorescence values detected (RFU) at times of 10 seconds, +5 minutes, +10 minutes, and +30 minutes are reported. The mean fluorescence values detected for the control wells were 476 RFU at 10 seconds; 392 RFU at +5 minutes; 355 RFU at +10 minutes, and 319 RFU at +30 minutes.

### Comparative Example D

The procedure of Example 5 was followed, with the exception that the culture media (pH 7.0) containing 6′-chloro-4′-methylumbelliferyl α-D-glucopyranoside (0.3 mg/mL) was replaced with culture media (pH 8.0) containing 4′-methylumbelliferyl α-D-glucopyranoside (0.3 mg/mL) (MUG). In Table 2, the mean fluorescence values detected (RFU) at times of 10 seconds, +5 minutes, +10 minutes, and +30 minutes are reported. The mean fluorescence values detected for the control wells were 432 RFU at 10 seconds; 347 RFU at +5 minutes; 302 RFU at +10 minutes, and 252 RFU at +30 minutes.

### Comparative Example E

The procedure of Example 5 was followed, with the exception that the culture media (pH 7.0) containing 6′-chloro-4′-methylumbelliferyl α-D-glucopyranoside (0.3 mg/mL) was replaced with culture media (pH 7.0) containing 8′-fluoro-4′-methylumbelliferyl α-D-glucopyranoside (0.3 mg/mL) (8F-MUG). In Table 2, the mean fluorescence values detected (RFU) at times of 10 seconds, +5 minutes, +10 minutes, and +30 minutes are reported. The mean fluorescence values detected for the control wells were 369 RFU at 10 seconds; 306 RFU at +5 minutes; 271 RFU at +10 minutes, and 230 RFU at +30 minutes.

TABLE 2

| | | | FLUORESCENCE RESULTS | | | |
|---|---|---|---|---|---|---|
| | | | WITH 5 × 10³ SPORES | | | |
| | pH of | ENZYME | MEASURED FLUORESCENCE, RFU | | | |
| | CULTURE | SUBSTRATE | 10 | +5 | +10 | +30 |
| EXAMPLE | MEDIA | USED | Sec | min | min | min |
| Example 5 | 7.0 | Example 1 | 1226 | 2377 | 4424 | 11941 |
| Example 6 | 7.0 | Example 2 | 1500 | 3370 | 6612 | 16521 |
| Comparative | 8.0 | MUG | 540 | 658 | 993 | 2891 |
| Example D | | | | | | |
| Comparative | 7.0 | 8F-MUG | 777 | 1573 | 3153 | 9038 |
| Example E | | | | | | |

### Example 7

Culture media (pH 6.7) containing 6′-chloro-4′-methylumbelliferyl α-D-glucopyranoside (0.3 mg/mL) was each added to eight wells (100 microliters per well) of a black 96-well microtiter plate with (NUNC #165301, Thermo Fisher Scientific, Waltham, Massachusetts). A 5-microliter aqueous suspension containing 5×10⁵ Geobacillus stearothermophilus spores (ATCC 7953) was added to 4 of the wells for each medium in the microtiter plate. Two control wells were also prepared that did not contain any Geobacillus stearothermophilus spores. The top of the plate was covered with an optically clear sealing film (product #4311971, Applied Biosystems Corporation, Foster City, California). The plate was warmed at 60° C. and shaken in a linear direction. Fluorescence readings (360 nm excitation/450 nm emission at 50% gain) were taken from the bottom of the plate. A reading was taken at an initial time point of 10 seconds and then at +1-minute intervals thereafter. A Synergy Neo2 fluorescence plate reader (BioTek Company, Winooski, Vermont) was used. In Table 3, the mean fluorescence values detected (RFU) at times of 10 seconds, +5 minutes, +10 minutes, and +30 minutes are reported. The mean fluorescence values detected for the control wells were 511 RFU at 10 seconds; 426 RFU at +5 minutes; 373 RFU at +10 minutes, and 310 RFU at +30 minutes.

### Example 8

The procedure of Example 7 was followed, with the exception that the culture media (pH 6.7) containing 6′-chloro-4′-methylumbelliferyl α-D-glucopyranoside (0.3 mg/mL) was replaced with culture media (pH 6.7) containing 8′-chloro-4′-methylumbelliferyl α-D-glucopyranoside (0.3 mg/mL). In Table 3, the mean fluorescence values detected (RFU) at times of 10 seconds, +5 minutes, +10 minutes, and +30 minutes are reported. The mean fluorescence values detected for the control wells were 428 RFU at 10 seconds; 365 RFU at +5 minutes: 330 RFU at +10 minutes, and 292 RFU at +30 minutes.

### Comparative Example F

The procedure of Example 7 was followed, with the exception that the culture media (pH 6.7) containing 6′-chloro-4′-methylumbelliferyl α-D-glucopyranoside (0.3 mg/mL) was replaced with culture media (pH 6.7) containing 4′-methylumbelliferyl α-D-glucopyranoside (0.3 mg/mL) (MUG). In Table 3, the mean fluorescence values detected (RFU) at times of 10 seconds, +5 minutes, +10 minutes, and +30 minutes are reported. The mean fluorescence values detected for the control wells were 402 RFU at 10 seconds; 335 RFU at +5 minutes; 297 RFU at +10 minutes, and 243 RFU at +30 minutes.

### Comparative Example G

The procedure of Example 7 was followed, with the exception that the culture media (pH 6.7) containing 6′-chloro-4′-methylumbelliferyl α-D-glucopyranoside (0.3 mg/mL) was replaced with culture media (pH 6.7) containing 8′-fluoro-4′-methylumbelliferyl α-D-glucopyranoside (0.3 mg/mL) (8F-MUG). In Table 3, the mean fluorescence values detected (RFU) at times of 10 seconds, +5 minutes, +10 minutes, and +30 minutes are reported. The mean fluorescence values detected for the control wells were 354 RFU at 10 seconds; 299 RFU at +5 minutes; 265 RFU at +10 minutes, and 224 RFU at +30 minutes.

TABLE 3

| | | | FLUORESCENCE RESULTS | | | |
|---|---|---|---|---|---|---|
| | | | WITH 5 × 10⁵ SPORES | | | |
| | pH of | ENZYME | MEASURED FLUORESCENCE, RFU | | | |
| | CULTURE | SUBSTRATE | 10 | +5 | +10 | +30 |
| EXAMPLE | MEDIA | USED | sec | min | min | min |
| Example 7 | 6.7 | Example 1 | 17227 | 53202 | 79375 | 98077 |
| Example 8 | 6.7 | Example 2 | 21302 | 56949 | 73987 | 81560 |
| Comparative | 6.7 | MUG | 895 | 3260 | 8049 | 29586 |
| Example F | | | | | | |
| Comparative | 6.7 | 8F-MUG | 8975 | 31069 | 50175 | 67041 |
| Example G | | | | | | |

### Example 9

Culture media (pH 7.0) containing 6′-chloro-4′-methylumbelliferyl □-D-glucopyranoside (0.3 mg/mL) was each added to eight wells (100 microliters per well) of a black 96-well microtiter plate with (NUNC #165301, Thermo Fisher Scientific, Waltham, Massachusetts). A 5-microliter aqueous suspension containing 5×10⁵ Geobacillus stearothermophilus spores (ATCC 7953) was added to 4 of the wells for each medium in the microtiter plate. Two control wells were also prepared that did not contain any Geobacillus stearothermophilus spores. The top of the plate was covered with an optically clear sealing film (product #4311971, Applied Biosystems Corporation, Foster City, California). The plate was warmed at 60° C. and shaken in a linear direction. Fluorescence readings (360 nm excitation/450 nm emission at 50% gain) were taken from the bottom of the plate. A reading was taken at an initial time point of 10 seconds and then at +1-minute intervals thereafter. A Synergy Neo2 fluorescence plate reader (BioTek Company, Winooski, Vermont) was used. In Table 4, the mean fluorescence values detected (RFU) at times of 10 seconds, +5 minutes, +10 minutes, and +30 minutes are reported. The mean fluorescence values detected for the control wells were 512 RFU at 10 seconds; 417 RFU at +5 minutes: 363 RFU at +10 minutes, and 308 RFU at +30 minutes.

### Example 10

The procedure of Example 9 was followed, with the exception that the culture media (pH 7.0) containing 6′-chloro-4′-methylumbelliferyl α-D-glucopyranoside (0.3 mg/mL) was replaced with culture media (pH 7.0) containing 8′-chloro-4′-methylumbelliferyl α-D-glucopyranoside (0.3 mg/mL). In Table 4, the mean fluorescence values detected (RFU) at times of 10 seconds, +5 minutes, +10 minutes, and +30 minutes are reported. The mean fluorescence values detected for the control wells were 476 RFU at 10 seconds; 392 RFU at +5 minutes: 355 RFU at +10 minutes, and 319 RFU at +30 minutes.

### Comparative Example H

The procedure of Example 9 was followed, with the exception that the culture media (pH 7.0) containing 6′-chloro-4′-methylumbelliferyl α-D-glucopyranoside (0.3 mg/mL) was replaced with culture media (pH 8.0) containing 4′-methylumbelliferyl α-D-glucopyranoside (0.3 mg/mL) (MUG). In Table 4, the mean fluorescence values detected (RFU) at times of 10 seconds, +5 minutes, +10 minutes, and +30 minutes are reported. The mean fluorescence values detected for the control wells were 432 RFU at 10 seconds; 347 RFU at +5 minutes: 302 RFU at +10 minutes, and 252 RFU at +30 minutes.

### Comparative Example I

The procedure of Example 9 was followed, with the exception that the culture media (pH 7.0) containing 6′-chloro-4′-methylumbelliferyl α-D-glucopyranoside (0.3 mg/mL) was replaced with culture media (pH 7.0) containing 8′-fluoro-4′-methylumbelliferyl α-D-glucopyranoside (0.3 mg/mL) (8F-MUG). In Table 4, the mean fluorescence values detected (RFU) at times of 10 seconds, +5 minutes, +10 minutes, and +30 minutes are reported. The mean fluorescence values detected for the control wells were 369 RFU at 10 seconds; 306 RFU at +5 minutes: 271 RFU at +10 minutes, and 230 RFU at +30 minutes.

TABLE 4

| | | | FLUORESCENCE RESULTS | | | |
|---|---|---|---|---|---|---|
| | | | WITH 5 × 10⁵ SPORES | | | |
| | pH of | ENZYME | MEASURED FLUORESCENCE, RFU | | | |
| | CULTURE | SUBSTRATE | 10 | +5 | +10 | +30 |
| EXAMPLE | MEDIA | USED | sec | min | min | min |
| Example 9 | 7.0 | Example 1 | 18386 | 53621 | 81061 | 99019 |
| Example 10 | 7.0 | Example 2 | 21883 | 58454 | 77112 | 83010 |
| Comparative | 8.0 | MUG | 2644 | 12059 | 29277 | 75861 |
| Example H | | | | | | |
| Comparative | 7.0 | 8F-MUG | 8171 | 34178 | 56663 | 70392 |
| Example I | | | | | | |

The examples provided above demonstrate that Examples 1 and 2 (6′-chloro-4′-methylumbelliferyl α-D-glucopyranoside and 8′-chloro-4′-methylumbelliferyl α-D-glucopyranoside, respectively) are easily prepared and exhibit a higher fluorescent signal in the presence of Geobacillus stearothermophilus spores than the traditional enzyme substrate 4′-methylumbelliferyl α-D-glucopyranoside (MUG), or even our previous invention 8′-fluoro-4′-methylumbelliferyl α-D-glucopyranoside. This has been demonstrated at high and low spore levels, at two low pH levels, and by comparison to MUG at its optimal pH of 8.0.

Any cited references, patents, and patent applications in this application that are incorporated by reference are incorporated in a consistent manner. In the event of inconsistencies or contradictions between portions of the incorporated references and this application, the information in this application shall control. The preceding description, given in order to enable one of ordinary skill in the art to practice the claimed disclosure, is not to be construed as limiting the scope of the disclosure, which is defined by the claims and all equivalents thereto.

1. A chlorinated 4′-alkylumbelliferyl α-D-glucopyranoside represented by the structural formula 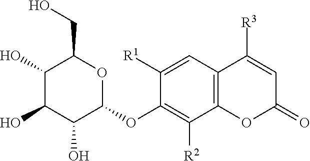
wherein:
one of R¹ and R² is Cl and the other is H; and
R³ represents an alkyl group having 1 to 12 carbon atoms.

2. The chlorinated 4′-alkylumbelliferyl α-D-glucopyranoside of claim 1, wherein R¹ is Cl and R² is H.

3. The chlorinated 4′-alkylumbelliferyl α-D-glucopyranoside of claim 1, wherein R² is Cl and R¹ is H.

4. The chlorinated 4′-alkylumbelliferyl α-D-glucopyranoside of claim 1, wherein R³ has from 1 to 4 carbon atoms.

5. The chlorinated 4′-alkylumbelliferyl α-D-glucopyranoside of claim 1, wherein R³ is methyl.

6. A method of assessing efficacy of a sterilization process, the method comprising sequentially:
a) providing a biological sterilization indicator comprising:
i) bacterial spores comprising, and/or capable of producing, an enzyme capable of catalyzing cleavage of an enzyme substrate represented by the structural formula 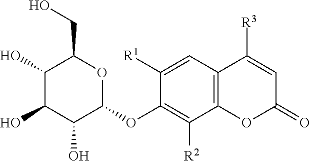
wherein:
one of R¹ and R² is Cl and the other is H, and
R³ represents an alkyl group having 1 to 12 carbon atoms; and
ii) a composition, wherein the composition comprises the enzyme substrate, wherein if the composition is brought into contact with the bacterial spores to form a mixture, the mixture will have an initial pH in the range from 5.5 to 9.0;
b) subjecting at least the bacterial spores to the sterilization process;
c) contacting the composition with the bacterial spores; and
d) evaluating efficacy of the sterilization process.

7. The method of claim 6, wherein step d) comprises fluorescence spectroscopy.

8. The method of claim 6, wherein R¹ is Cl and R² is H.

9. The method of claim 6, wherein R² is Cl and R¹ is H.

10. The method of claim 6, wherein R is an alkyl group having 1 to 4 carbon atoms.

11. The method of claim 6, wherein R is methyl.
